(19) United States
(12) Patent Application Publication
(10) Pub. No.: US 2015/0031030 A1
(43) Pub. Date: Jan. 29, 2015

(54) METHOD FOR ANALYSING FOETAL NUCLEIC ACIDS

(71) Applicant: IVF Zentren Prof. Zech-Bregenz GmbH, Bregenz (AT)

(72) Inventor: Nicolas Zech, Bregenz (AT)

(73) Assignee: IVF ZENTREN PROF. ZECH - BREGENZ GMBH, Bregenz (AT)

(21) Appl. No.: 14/377,909

(22) PCT Filed: Feb. 8, 2013
(86) PCT No.: PCT/AT2013/050033

§ 371 Date: Oct. 8, 2014

(30) Foreign Application Priority Data
Feb. 10, 2012 (AT) A 178/2012

(51) Int. Cl. C12Q 1/68

(52) U.S. Cl. CPC C12Q 1/6883; C12Q 1/6881; C12Q 1/6879; C12Q 1/6806; C12Q 2600/156; C12Q 2600/124
USPC 435/6.11; 435/6.12; 435/6.1

(57) ABSTRACT

The invention relates to a method for analyzing fetal nucleic acids from the supernatant of the culture medium of fetal cell cultures of an in vitro fertilization, comprising the steps: a) removing from the culture vessel used for cultivating the fertilized egg cell the cell-free supernatant of the in vitro fertilization culture medium, which supernatant contains fetal nucleic acid, b) enriching the fetal nucleic acids contained in the supernatant, and c) carrying out an analysis of the nucleic acids present in the cell-free supernatant. The invention further relates to a method for analyzing fetal nucleic acids from the liquid surrounding the embryo within the zona pellucida, the blastocoel or the blastocyst cavity, comprising the steps: a) removing the fluid containing the cell-free fetal nucleic acid from the liquid surrounding the embryo within the zona pellucida, the blastocoel or the blastocyst cavity of the blastula or blastocyst, b) enriching the nucleic acid contained in the fluid surrounding the embryo within the zona pellucida, or the fetal nucleic acids contained in the blastocoel or the blastocyst cavity, and c) carrying out an analysis of the nucleic acids present in the cell-free supernatant.

The invention relates to a method for analyzing fetal nucleic acids from the supernatant of the culture medium of fetal cell cultures or from the fluid surrounding the embryo within the egg cell membrane (zona pellucida), from the blastocoel or from the blastocyst cavity.

Cellular fetal DNA can be analyzed both during the pre-implantation diagnosis (PID), which relates to the examination of the embryo prior to implantation into the uterus, and also during the pre-natal diagnosis (PND) which relates to the examination of the fetus before birth.

Invasive methods are available for prenatal diagnosis, such as chorionic villus sampling, amniocentesis and umbilical cord puncture, for examining the genetic information of the developing fetus as well as non-invasive methods, such as ultrasound examinations.

Invasive methods require the insertion of a needle into the uterus in order to extract either amniotic fluid or tissue from the placenta or blood from the fetus. This may cause complications in the pregnancy which could lead to miscarriage.

The possibility of recovering and analyzing genetic material from the fetus using non-invasive methods can eliminate the risk of harm to the health of the fetus and of an unwanted termination of the pregnancy. The blood plasma of the mother contains freely circulating DNA of small molecular size in addition to cellular, maternal nucleic acids. A proportion of this freely circulating DNA in the blood plasma of pregnant women originates from the fetus. In addition to ultrasound examinations non-invasive, prenatal molecular diagnosis is based on the analysis of this freely circulating fetal DNA. The percentage of fetal DNA in the total amount of DNA in the maternal blood plasma is about three to six percent and increases over the course of the pregnancy. In the first trimester the concentration of fetal DNA is on average about 25 genome equivalents (GE) per milliliter of plasma and increases to about 300 GE/ml in the third trimester. The individual concentrations of fetal DNA in the plasma vary up to about tenfold. After birth the level of fetal DNA in the blood plasma drops rapidly and after 24 hours freely circulating fetal DNA sequences are already no longer evident in the blood of the mother and are thus completely eliminated before a further pregnancy. The origin of freely circulating DNA is presumed to be apoptotic processes in the placenta, which cause the release of degraded genomic DNA. In this way DNA from the fetus is continually released into the bloodstream of the mother. Several studies have shown that in the plasma of pregnant women fetal and maternal DNA differ in their molecular properties: fetal DNA is mostly in the form of short to 300 base pairs (bp) of larger fragments, whereas maternal DNA mainly circulates in a higher molecular form (1000 by and above). The difference in size between fetal and maternal cell-free DNA remains largely unchanged over the course of the pregnancy and makes it possible to enrich short DNA fragments using suitable methods of DNA purification and thus obtain a DNA sample in which the proportion of fetal DNA is increased relative to the dominant background of maternal DNA sequences. This enrichment of fetal DNA increases the value of the information provided by the molecular diagnosis of the fetus for detecting sequence changes/mutations and in particular quantitative information, which is used for diagnosing trisomies.

However, evidence of fetal DNA in the maternal blood plasma does not enable the molecular genetic analysis of the embryo during in vitro fertilization (IVF) prior to transferring the embryo into the uterus of the mother.

However, in order to increase the success rate of in vitro fertilization, the identification of the best embryos prior to transfer in terms of morphology and also molecular genetics is increasingly important, which is possible by means of pre-implantation diagnosis.

Cell biological and molecular genetic investigations are referred to as pre-implantation diagnosis (PID) and are used to decide whether an embryo produced by in vitro fertilization should be implanted into the uterus or not. PID is mainly used for identifying specific hereditary diseases and special features of the chromosomes. It can however also be used for making a baby that will be able to donate an organ to a sick sibling (savior sibling) or for selecting the sex or specific hereditary characteristics of the child.

In order to perform a PID it is necessary for in vitro fertilization to take place. The method of IVF with PID can be divided into roughly five steps: 1. hormone stimulation and recovery of egg cells, 2. in vitro fertilization, 3. embryo biopsy, 4. genetic diagnosis, 5. embryo transfer or cryopreservation. Steps three and four constitute the PID in the narrower sense.

The embryo biopsy, i.e. the separation of one or two cells from an embryo, is generally performed on the third day after fertilization. The embryo usually consists at this time of six to ten cells and is surrounded by a protective membrane (zona pellucida).

According to a newer method the embryo is only biopsied on about the fifth day of development. At this stage of development the embryo consists of an outer cell group, from which the placenta develops (trophoblast), and the inner cell mass, from which the embryo or fetus develops (embryoblast) and is referred to as a blastocyst. This is thus also referred to as blastocyst biopsy. In a blastocyst biopsy generally several cells are taken from the trophoblast and examined genetically. Only about 20-40% of all fertilized egg cells reach the stage of the expanded blastocyst.

In an embryo biopsy firstly an opening is made in the protective membrane surrounding the embryo by means of acid, laser light or by mechanical means. Afterwards one or two cells are removed from the embryo by means of a pipette. There are indications that the separation of cells may possibly reduce the implantability of the embryo. It has not been fully explained whether the separation could also have further negative effects on the development of the embryo or the child. PID is a difficult process to perform, not least because there are usually at most only two cells available for the test and the process cannot be repeated or can only be repeated with difficulty. Therefore, the risk of misdiagnosis cannot be ignored. The probability of a correct test result is about 90-95%. To check the result all affected couples are advised to undergo a PND during pregnancy. The most common problem is false negative results which are caused by contamination with foreign DNA or by so-called “allelic dropout”, i.e. the analysis of only one instead of both alleles. In the case of a false negative test result the embryo is a carrier of the genetic defect even though the diagnosis does not reveal this.

An additional problem is caused by mosaicism, a mosaic being defined as an embryo which is made up of genetically different cells. It may be the case that the examined cells have a different genome from the remaining cells, which results in misdiagnosis. Mosaicism occurs relatively frequently and is the result of faults that occur during cell division.

The objective of the present invention is therefore to enable pre-implantation diagnosis that does not endanger the embryo but still provides reliable information.

The objective of the invention is achieved independently by a method for analyzing fetal nucleic acids from the supernatant of the culture medium of fetal cells cultures of in vitro fertilization comprising the steps: a) extracting cell-free supernatant containing fetal nucleic acid from the in vitro fertilization culture medium from the culture vessel used for cultivating the fertilized egg cell, b) enriching the fetal nucleic acids contained in the supernatant and c) performing an analysis of the nucleic acids present in the cell-free supernatant, and by a method for analyzing fetal nucleic acids from the fluid surrounding the embryo within the zona pellucida, the blastocoel or the blastocyst cavity comprising the steps: a) extracting the cell-free fluid containing fetal nucleic acid from the fluid surrounding the embryo within the zona pellucida, the blastocoel or the blastocyst cavity of the blastula or blastocyst, b) enriching the nucleic acid contained in that fluid surrounding the embryo within the zona pellucida, or the fetal nucleic acids contained in the blastocoel or blastocyst cavity and c) performing an analysis of the nucleic acids present in the cell-free supernatant.

The use of cell-free fetal DNA from the supernatant of the culture medium of in vitro cultures from in vitro fertilization instead of intact cells has the advantage that a) the implantatability of the embryo is not reduced, b) the risk of unwanted termination is avoided, c) the embryo is not damaged, d) faults caused by mosaicism, microchimerism and contamination with foreign DNA are reduced, e) misdiagnoses are avoided, etc.

It has shown to be advantageous in the method according to the invention that no cellular components have to be removed. By means of the non-invasive preimplantation diagnosis fetal chromosomal aneuploidies, monogenetic diseases, the sex, blood group and HLA typing etc. can be established.

Furthermore, it has proved to be an advantage that by using molecular genetic methods the result can be provided rapidly and thus it is possible to make a decision prior to the transfer of the embryo into the uterus of the mother, which usually takes place at the latest 6 days after fertilization, if cryopreservation is not performed.

The fetal nucleic acid can be DNA and/or RNA, in particular genomic or mitochondrial DNA, and it has proved to be an advantage that standardized molecular biological methods can be used for subsequent analysis.

It is also an advantage that the cell culture supernatant of mono and/or sequential culture media can be analyzed, whereby the analysis can be repeated during the preimplantation diagnosis if the result is uncertain, as at any time fetal nucleic acids can be isolated non-invasively from the cell culture and analyzed.

The cell-free supernatant containing nucleic acid can be analyzed on days 1 to 6 of the in vitro culture, whereby the result of the analysis is provided very early during the in vitro fertilization. Thus for example future parents have a basis for making a decision about a further course of action prior to implantation without putting the embryo at risk. Preferably, the cell-free fetal DNA/RNA is analyzed on days 3 to 5 of the in vitro culture, as at this time there is already sufficient fetal DNA/RNA of the embryo in the cell culture supernatant of the in vitro cell culture in order to obtain a reliable result from the analysis and also the result is available before the transfer of the embryo into the uterus of the mother.

Preferably, the cell culture supernatant is extracted from an area surrounding the fertilized egg cell that is at least twice the diameter of the cultivated fertilized egg cell, because the concentration of fetal DNA/RNA is greatest in this area and thus the probability of obtaining a result from the analysis is greatest.

It is also possible to open the zona pellucida of the embryo or the fertilized egg cell on culture days 1 to 6 of the cell culture prior to extracting the cell culture supernatant mechanically, chemically or by laser, in order to increase the concentration of fetal DNA/RNA, as the zona pellucida is a natural barrier for the escape of DNA/RNA into the culture medium.

Depending on the method (without or after opening the zona pellucida) at least 2 μl of the cell culture supernatant is analyzed, as in this way sufficient fetal DNA/RNA can be provided for analysis.

The isolation, in particular enrichment, of the fetal nucleic acid is performed by a method selected from a group comprising centrifugation, filtration, binding to beads and amplification, in particular a polymer chain reaction, as in this way standardized methods are used to obtain the result and thus the error rate can be kept as low as possible.

The analysis of fetal nucleic acids can comprise a method selected from a group containing sequencing, amplification and in situ hybridization, in particular fluorescence in situ hybridization, whereby a meaningful result is provided rapidly and it is possible to make a decision about the fate of the embryo prior to embryo transfer.

It has proved to be an advantage that diseases, in particular monogenetic diseases, blood groups, the sex, aneuploidies and HLA types can be detected and thus the original problem can be addressed prior to the beginning of the actual pregnancy and not just during the pregnancy after a long waiting period or by endangering the embryo.

In a method for analyzing fetal nucleic acids from the fluid surrounding the embryo within the zona pellucida, the blastocoel or blastocyst cavity which comprises the steps: a) extracting the cell-free fluid containing fetal nucleic acid from the fluid surrounding the embryo within the zona pellucida, the blastocoel or the blastocyst cavity of the blastula or blastocyst, b) enriching the fetal nucleic acids contained in the fluid surrounding the embryo within the zona pellucida, in the blastocoel or blastocyst cavity and c) performing an analysis of the nucleic acids present in the cell-free supernatant, a quantity of fluid selected from a range with a lower limit of 0.004 pl and an upper limit of 0.1 nl, in particular 0.04 nl, is extracted from the fluid surrounding the embryo within the zona pellucida, from the fluid contained in the blastocoel or the blastocyst cavity of the blastula or blastocyst and transferred into a fluid volume, preferably consisting of or containing the culture medium, of at least 2 μl and is subsequently analyzed, so as not to damage the embryo, the blastula or blastocyst and also providing sufficient material for further analysis.

The isolation and enrichment of the nucleic acid is performed by amplification, in particular a polymer chain reaction, as in this way a sufficient amount of fetal nucleic acid is provided rapidly so that it can be presented for further analysis.

The advantages of isolating and analyzing the nucleic acids using standardized methods correspond to the ones described above, even in this alternative embodiment of the method, where the cell-free nucleic acids are extracted from the fluid surrounding the embryo within the zona pellucida, the blastocoel or the blastocyst cavity. In addition, by means of the alternative method according to the invention the same parameters can be established and determined as described above.

For a better understanding of the invention and the latter is explained in more detail with reference to the following FIGURE.

In a diagrammatic much simplified representation:

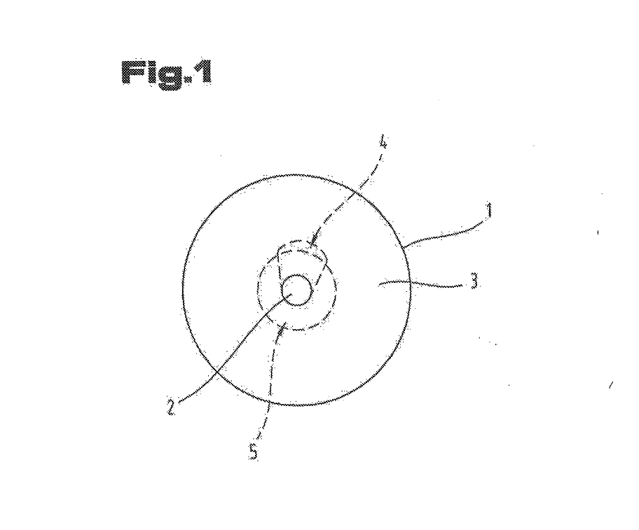

FIG. 1 shows a culture vessel with a fertilized egg cell and the surrounding area.

First of all, it should be noted that in the variously described embodiments the same parts have been given the same reference numerals, whereby the disclosures contained throughout the entire description can be applied to the same parts with the same reference numerals. Also details relating to position used in the description, such as e.g. top, bottom, side etc. relate to the currently described and represented FIGURE and in case of a change in position should be adjusted to the new position. Furthermore, also individual features or combinations of features from the various exemplary embodiments shown and described can represent in themselves independent or inventive solutions.

All of the details relating to value ranges in the present description are defined such that the latter include any and all part ranges therefrom, e.g. a range of 1 to 10 means that all part ranges, starting from the lower limit of 1 to the upper limit 10 are included, i.e. the whole part range beginning with a lower limit of 1 or above and ending at an upper limit of 10 or less, e.g. 1 to 1.7, or 3.2 to 8.1 or 5.5 to 10.

The present invention describes a method, by means of which pre-implantation diagnosis can be performed without using cellular material.

An embryo is defined within the meaning of the invention to be the cellular part of the in vitro culture from which the actual embryo develops including the early stages of development, such as the morula, blastula, gastrula or blastocyst.

The fertilized egg cell within the meaning of the invention represents the various early stages of development of embryogenesis, such as the morula, blastula, gastrula or blastocyst.

To perform the method of the invention for analyzing fetal nucleic acids from the supernatant of the culture medium of fetal cells cultures of an in vitro fertilization cell culture, firstly an aliquot of the culture medium supernatant or the whole culture medium supernatant is extracted from the culture vessel used for cultivating the fertilized egg cell, which is cell-free and contains fetal nucleic acids. Afterwards the culture medium containing the nucleic acid is processed further and the fetal nucleic acids contained in the supernatant are isolated. Said isolated nucleic acid of the cell-free supernatant is then subjected to further analysis.

Standardized media, such as HSA or rHSA media, are used as the culture medium for the fertilized egg cell or the embryo, whereby no foreign DNA is introduced into the cells cultures.

However, before the fertilized egg cell can be cultivated firstly the egg cells are removed (follicular puncture) and then the egg cell is fertilized with the sperm cell by means of IVF, ICSI or IMSI. The thus fertilized egg cells are placed into a culture medium, also known as a growing medium. The latter are then incubated in an incubator. After 16 to 18 hours a first check is performed under a microscope to establish how many egg cells have been fertilized by the sperm cells. It is possible in the meantime to keep fertilized egg cells or embryos in the culture for longer and then on day 5 (rarely on day 6) after follicular puncture to transfer one or two embryos in the blastocyst stage, preferably in the expanded blastocyst stage.

This means that out of all of the embryos cultivated for 3 to 6 days, the most developed embryos and those that are morphologically the least conspicuous and least noticeable in terms of molecular genetics according the method of the invention are selected and transferred.

Surprisingly there is a sufficient amount of extracellular fetal nucleic acid in the fluid surrounding the embryo within the zona pellucida, in the blastocoel or the blastocyst cavity and in the culture medium supernatant for it to be subjected to further analysis. It is assumed that the DNA comes from apoptotic cells inside the embryo. The DNA from these cells is released and can pass through the zona pellucida. Therefore, in order to perform a molecular-genetic analysis of the embryo intended for transfer the removal of cellular material is made obsolete by the method according to the invention. Thus no cells of any kind need to be removed, not from the later embryo nor the cells surrounding it nor from the morula, blastula, gastrula or blastocyst stage. As the embryo to be developed is in a very active stage in contrast to the maternal egg cell and the paternal sperm cell, the amount of fetal nucleic acid in the cell-free culture medium is much higher than that of the parents.

By means of the cell-free recovery of the nucleic acid from the supernatant the further processing of the latter for performing an analysis can be simplified significantly, as no cellular components have to be separated in which process fetal nucleic acids would possibly be lost, and there are no cellular impurities which would possibly interfere with the analysis.

The fetal nucleic acid can be both DNA and RNA, but mostly consists of nucleic acid fragments. Thus freely circulating DNA fragments with a small molecular size can be analyzed. However, RNA, such as e.g. miRNA, siRNA or mRNA, which is firstly reverse transcribed for example for further analysis, can also be isolated and analyzed.

The supernatant of the culture medium to be analyzed can be analyzed from mono or sequential culture media. When using sequential culture media during the cultivation of the fertilized egg cell the culture medium is possibly changed several times, whereby with each change of culture medium the fetal nucleic acids can be isolated and analyzed from the extracted culture medium. However, a multiple change of the culture medium is also possible even with mono culture media.

The cell-free supernatant containing the nucleic acid is extracted on at least one of days 1 to 6 of the in vitro culture and analyzed. Preferably, the supernatant is extracted between day 3 and 5 of the in vitro culture (without or after mechanical, chemical or laser opening of the zona pellucida), because in this time period there are sufficient fetal nucleic acids in the supernatant and the result of the pre-implantation diagnosis can still be provided in good time prior to the transfer of the embryo into the uterus of the mother. The opening of the zona pellucida can be performed immediately before extracting the supernatant of the culture medium or even up to 3 days before.

If the culture medium is not changed or fetal nucleic acid is analyzed before a culture medium change, the cell culture supernatant is extracted from an area surrounding the fertilized egg cell of at least twice the diameter of the cultivated fertilized egg cell. In FIG. 1, the area from which the culture medium can be extracted is not represented to scale. FIG. 1 shows a culture vessel 1 in which the fertilized egg cell 2 is being cultivated in the culture medium 3. A possible area 4 surrounding the fertilized egg cell 2 borders the fertilized egg cell 2 and extends to the upper edge thereof. A further option for area 5 is also shown in FIG. 1, the latter extending radially around the egg cell 2. In order to recover a sufficient amount of fetal nucleic acid, preferably the volume is extracted from the distance of twice the maximum diameter of the fertilized egg cell 2.

At least 2 μl of the cell culture supernatant is extracted in order to ensure that it contains at least a few molecules of fetal nucleic acid, which are generally amplified before being subjected to further analysis.

However, during a change of culture medium or at the time of embryo transfer into the uterus the whole culture medium can also be subjected to further analysis.

The isolation of fetal nucleic acids from the cell-free cell culture supernatant is performed by methods known from the prior art which are used for enriching nucleic acids. The latter are in particular methods selected from a group comprising centrifugation, filtration, binding to beads and amplification, in particular a polymer chain reaction, etc.

However the presence and amount of fetal DNA can also be established without enrichment or isolation, for example by spectrophotometric methods. For example in this way the amount of nucleic acids in the sample (as DNA or RNA) can be calculated even from a small sample (1 μl) and the obtained spectrum. From the latter indications relating to the further development of the fertilized egg cell or embryo can be acquired.

The analysis of the fetal nucleic acids is also performed by way of methods known from the prior art, in which preferably a method is performed that is selected from a group comprising sequencing, amplification and in situ hybridization, in particular fluorescence in situ hybridization. The examination of the nucleic acids is performed, depending on the underlying problem, by means of various different analysis methods and usually takes between two and 24 hours. For example, the amplification and if necessary isolation of the nucleic acid can be performed using amplification methods, whereby quantities of a cell equivalent DNA can be analyzed by means of whole genome amplification. For example the FisH (Fluorescence in situ Hybridization) test tests for chromosome aberrations, very significant changes in the genome. Individual genes are examined if the parents have a predisposition to a genetic defect, if a specific hereditary disease frequently occurs in the family. In addition to the most frequently used methods of PCR amplification and fluorescence in situ hybridization, methods known from the prior art of “whole gene amplification” are also available for preimplantation diagnosis. In addition, “karyomapping” can be performed as a possible method of analysis for the nucleic acids recovered by the method of the invention.

From the analysis of cell-free genomic or mitochondrial DNA of an embryo in the culture important parameters can be established relating to the chances of success of the implantation, the viability and the genetic predisposition of the developing living being. It is possible to detect diseases, in particular monogenetic diseases, blood groups, the sex, aneuploidies and HLA types, etc. However, other parameters can also be determined on a molecular genetic basis. By means of the method according to the invention biological markers can also be detected which are associated with chromosomal aneuploidies and have a molecular genetic source.

In an alternative embodiment variant of the invention a method for analyzing fetal nucleic acid from the fluid surrounding the embryo within the zona pellucida, the blastocoel or the blastocyst cavity can also be used for pre-implantation diagnosis without using cellular material. In a first step cell-free fluid containing fetal nucleic acid from the fluid surrounding the embryo within the zona pellucida, the blastocoel or the blastocyst cavity of the blastula or blastocyst is extracted. In the following step the fluid surrounding the embryo within the zona pellucida, in the blastocoel or the blastocyst cavity containing fetal nucleic acids is enriched or isolated. Lastly, a molecular-biological analysis of the nucleic acids in the cell-free supernatant is performed.

For example, the zona pellucida can be opened on day 3 and the culture medium collected on days 3 to 5. Alternatively, on day 3 the inner part can also be rinsed and the medium collected.

Samples can be taken from the blastocoel or the blastocyst cavity on day 5.

From the fluid surrounding the embryo within the zona pellucida, the blastocoel or the blastocyst cavity at least 0.004 pl, in particular 0.004 nl to 0.04 nl, fluid is extracted. Preferably, from the fluid surrounding the embryo within the zona pellucida at least 0.004 nl is taken and from the blastocoel or the blastocyst cavity the blastula or blastocyst at least 0.04 pl to 0.04 nl fluid is extracted and transferred into a fluid volume, ideally consisting of or containing the culture medium, of at least 2 μl and then analyzed, in order in any case to include molecules of fetal nucleic acids in the extracted fluid of the blastocoel or the blastocyst cavity or the fluid area surrounding the embryo within the zona pellucida.

The nucleic acid contained in the fluid of the blastocoel or the blastocyst cavity is preferably enriched by amplification, in particular a polymer chain reaction.

The aforementioned methods of isolation and analysis can be used and the same parameters determined.

The exemplary embodiments of the method for analyzing fetal nucleic acids from the supernatant of the culture medium of fetal cell cultures of in vitro fertilization are not restricted to the embodiment variants shown in particular, but rather various different combinations of the individual embodiment variants are also possible and this variability, due to the teaching on technical procedure, lies within the ability of a person skilled in the art in this technical field. Thus all conceivable embodiment variants, which are made possible by combining individual details of the embodiment variants shown and described, are also covered by the scope of protection.

Finally, as a point of formality, it should be noted that for a better understanding the components of FIG. 1 have not been represented true to scale in part and/or have been enlarged and/or reduced in size.

The underlying objective addressed by the independent solutions according to the invention can be taken from the description.

### LIST OF REFERENCE NUMERALS

1 culture vessel

2 egg cell

3 culture medium

4 area

5 area

1. A method for analyzing fetal nucleic acids from the supernatant of the culture medium of fetal cell cultures of in vitro fertilization comprising the steps: a) extracting cell-free supernatant containing fetal nucleic acid from the in vitro fertilization culture medium from the culture vessel used for cultivating the fertilized egg cell, b) isolating the fetal nucleic acids contained in the supernatant and c) performing an analysis of the nucleic acids present in the cell-free supernatant.

2. The method as claimed in claim 1, wherein DNA and/or RNA is analyzed as fetal nucleic acid.

3. The method as claimed in claim 1, wherein the cell culture supernatant of mono and/or sequential culture media is analyzed.

4. The method as claimed in claim 1, wherein the cell-free supernatant containing the nucleic acid is analyzed on at least one of days 1 to 6 of the in vitro culture.

5. The method as claimed in claim 1, wherein the supernatant of the culture medium (3) of the culture vessel (1) is extracted from an area (4, 5) surrounding the fertilized egg cell (2) which has at least twice the diameter of the cultivated fertilized egg cell (2).

6. The method as claimed in claim 1, wherein at least 2 μl of the cell culture supernatant is analyzed.

7. The method as claimed in claim 1, wherein the egg cell membrane (zona pellucida) of the fertilized egg cell is opened mechanically, chemically or by laser 1 to 6 days prior to the extraction of the cell culture supernatant.

8. The method as claimed in claim 1, wherein the isolation of the fetal nucleic acids is performed by enrichment, in particular by a method selected from a group comprising centrifugation, filtration, binding to beads and amplification, in particular a polymer chain reaction.

9. The method as claimed in claim 1, wherein the analysis of the fetal nucleic acids comprises a method selected from a group comprising sequencing, amplification and in situ hybridization, in particular fluorescence in situ hybridization.

10. The method as claimed in claim 1, wherein diseases, in particular monogenetic diseases, blood groups, the sex, aneuploidies and HLA types of the embryo are detected.

11. The method for analyzing fetal nucleic acids from the fluid surrounding the embryo within the egg cell membrane (zona pellucida), from the blastocoel or from the blastocyst cavity comprising the steps: a) extracting the cell-free fluid containing fetal nucleic acid from the fluid surrounding the embryo within the egg cell membrane (zona pellucida), the blastocoel or the blastocyst cavity of the blastula or blastocyst, b) enriching the fetal nucleic acids contained in that fluid surrounding the embryo within the egg cell membrane (zona pellucida), in the blastocoel or the blastocyst cavity and c) performing an analysis of the nucleic acids present in the cell-free supernatant.

12. The method as claimed in claim 11, wherein a quantity of fluid selected from a range with a lower limit of 0.004 pl, in particular 0.004 nl, and an upper limit of 0.1 nl is removed from the fluid surrounding the embryo within the egg cell membrane (zona pellucida), from the fluid contained in the blastocoel or blastocyst cavity of the blastula or blastocyst and transferred into a fluid volume, preferably consisting of or containing the culture medium, of at least 2 μl and then analyzed.

13. The method as claimed in claim 11, wherein the enrichment of the nucleic acid is performed by amplification, in particular a polymer chain reaction.

14. The method as claimed in claim 11, wherein the isolation of the fetal nucleic acids is performed by enrichment, in particular by a method selected from a group comprising centrifugation, filtration, binding to beads and amplification, in particular a polymerase chain reaction.

15. The method as claimed in claim 11, wherein the analysis of the fetal nucleic acids comprises a method selected from a group comprising sequencing, amplification and in situ hybridization, in particular fluorescence in situ hybridization.

16. The method as claimed in claim 11, wherein diseases, in particular monogenetic diseases, blood groups, the sex, aneuploidies and HLA types of the embryo are detected.
